(19) United States
(12) Patent Application Publication
(10) Pub. No.: US 2026/0137642 A1
(43) Pub. Date: May 21, 2026

(54) METHODS OF TREATING PATIENTS HAVING TYPE 1 DIABETES WITH EFLORNITHINE

(71) Applicants: CANCER PREVENTION PHARMACEUTICALS, INC., Waconia, MN (US); THE TRUSTEES OF INDIANA UNIVERSITY, Bloomington, IN (US)

(72) Inventors: Eugene GERNER, Tucson, AZ (US); Linda DIMEGLIO, Bloomington, IN (US); Raghavendra G. MIRMIRA, Bloomington, IN (US)

(73) Assignees: CANCER PREVENTION PHARMACEUTICALS, INC., Waconia, MN (US); THE TRUSTEES OF INDIANA UNIVERSITY, Bloomington, IN (US)

(21) Appl. No.: 18/705,908

(22) PCT Filed: Oct. 31, 2022
(86) PCT No.: PCT/US2022/078959

§ 371 Date: Apr. 29, 2024

Related U.S. Application Data
(60) Provisional application No. 63/274,654, filed on Nov. 2, 2021.

(51) Int. Cl. A61K 31/198; A61P 3/10; C12Q 1/6883

(52) U.S. Cl. CPC A61K 31/198; A61P 3/10; C12Q 1/6883; C12Q 2600/106; C12Q 2600/156

(57) ABSTRACT

Provided are methods for treating patients with type 1 diabetes, wherein the patient has new onset type 1 diabetes no more than eight months before starting treatment, and has not previously received an immunomodulatory agent. Also provided are methods for improving cell health in a patient having type 1 diabetes. Also provided are methods for preserving residual C-peptide in a patient having type 1 diabetes. The methods comprise administering an effective amount of a pharmaceutical therapy that comprises eflornithine while the patient is maintained on a low polyamine diet, wherein the method prevents, delays, decreases the likelihood of, or decreases the severity of diabetic ketoacidosis severe hypoglycemia, progression of diabetic nephropathy and retinopathy.

### REFERENCE TO RELATED APPLICATIONS

The present application claims the priority benefit of U.S. provisional application No. 63/274,654, filed Nov. 2, 2021, the entire contents of which is incorporated herein by reference.

### BACKGROUND

### 1. Field

The present invention relates generally to the fields of medicine and endocrinology. More particularly, it concerns methods for treating patients having type 1 diabetes.

### 2. Description of Related Art

Type 1 diabetes (T1D) develops through a cascade of steps leading to β cell destruction. Each step represents a possible valid target for altering disease progression. Although targeting a single pathway to enhance residual β cell function is unlikely to provide complete β cell recovery in T1D, even modest residual insulin production may be protective both for acute complications of T1D (such as diabetic ketoacidosis and severe hypoglycemia) and for longer-term microvascular disease (nephropathy and retinopathy). Therefore, therapies that even modestly improve β cell function in patients with T1D are needed.

### SUMMARY

In one embodiment, provided herein are methods of treating a patient having type 1 diabetes, the methods comprising administering to the patient a pharmaceutical therapy comprising an effective amount of eflornithine. In one embodiment, provided herein are compositions comprising a pharmaceutically effective amount of eflornithine for use in treating a patient having type 1 diabetes. In one embodiment, provided herein are uses of eflornithine in the manufacture of a medicament for the treatment of type 1 diabetes.

In some aspects, the methods or uses improve β cell health in the patient or prevent β cell apoptosis in the patient. In some aspects, the methods or uses preserve residual C-peptide in the patient.

In some aspects, the methods or uses prevent, delay, decrease the likelihood of, or decrease the severity of diabetic ketoacidosis in the patient. In some aspects, the methods or uses prevent, delay, or slow the progression of severe hypoglycemia, diabetic nephropathy, or diabetic retinopathy in the patient.

In some aspects, the patient is maintained on a low polyamine diet.

In some aspects, the patient has new onset type 1 diabetes. In some aspects, the patient was diagnosed with type 1 diabetes no more than eight months before starting treatment with eflornithine.

In some aspects, the patient has not previously received an immunomodulatory agent. In some aspects, the patient's random C-peptide level is greater than 0.2 pmol/mL. In some aspects, the patient is an adult. In some aspects, the patient is a pediatric patient. In some aspects, the patient is human.

In some aspects, the patient's genotype at position +316 (rs2302615) of at least one allele of the ODC1 gene has been determined. In some aspects, the patient's genotype has been determined to have a G at position +316 (rs2302615) of at least one allele of the ODC1 gene. In some aspects, the patient's genotype has been determined to have a G at position +316 (rs2302615) of both alleles of the ODC1 gene. In some aspects, the patient's genotype has been determined to have a G at position +316 (rs2302615) of one allele of the ODC1 gene and an A at position +316 (rs2302615) of one allele of the ODC1 gene. In some aspects, the methods or uses prevent ototoxicity or reduce the risk thereof within the patient.

In some aspects, the eflornithine is eflornithine hydrochloride. In some aspects, the eflornithine hydrochloride is eflornithine hydrochloride monohydrate. In some aspects, the eflornithine hydrochloride monohydrate is a racemic mixture of its two enantiomers. In some aspects, the eflornithine hydrochloride monohydrate is a substantially optically pure preparation.

In some aspects, the eflornithine is administered systemically. In some aspects, the eflornithine is administered orally, intraarterially or intravenously. In some aspects, the eflornithine is administered orally. In some aspects, the eflornithine is formulated for oral administration. In some aspects, the eflornithine is formulated as a hard or soft capsule or a tablet.

In some aspects, the effective amount of eflornithine is 125-1500 mg/m²/day. In some aspects, the effective amount of eflornithine is 750 mg/m²/day. In some aspects, the effective amount of eflornithine is 1000 mg/m²/day. In some aspects, the eflornithine is administered every 12 hours. In some aspects, the eflornithine is administered every 24 hours. In some aspects, the eflornithine is administered at least a second time.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Cellular Polyamine Homeostasis. Put 1: putrescine, Spd 2: spermidine, Spm 3: Spermine, ODC: ornithine decarboxylase, dcSAM: decarboxylated S-adenosylmethionine, N¹-acetylspermine 4, N1AcSpd 5: N¹-acetylspermidine, N8AcSpd 6: N⁸-acetylspermidine, SSAT: spermidine/spermine acetyltransferase, PAO: polyamine oxidase, PTS: polyamine transport system, PTI: polyamine transport inhibitor, 7: hydrogen peroxide, 8: amidoaldehyde.

### DETAILED DESCRIPTION

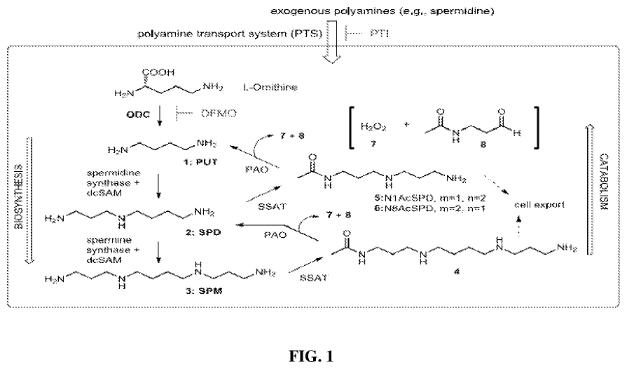

The polyamines (putrescine, spermidine, and spermine) are organic polycationic, low molecular weight aliphatic amines that are ubiquitous within living cells and important in governing the growth, proliferation, and survival of virtually all mammalian cell types. Importantly, and of relevance to the pathophysiology of type 1 diabetes (T1D), increases in cellular polyamine levels increase oxidative stress. Intracellular polyamine levels are regulated by: (a) endogenous biosynthesis (controlled by the rate-limiting enzyme ornithine decarboxylase (ODC), (b) uptake of exogenous polyamines through polyamine transporters, and (c) degradation processes (see FIG. 1). Because of their degree of protonation at physiological pH, the polyamines exist as linked cationic arrays (polycations) in vivo. Not surprisingly, polyamines interact with nucleic acids and can influence the structure of chromatin, gene transcription, DNA replication, and t-RNA formation as well as the function of membrane phospholipids, and ion channels (Brooks, 2012).

Polyamines can be derived endogenously from the amino acid ornithine (FIG. 1), which itself is produced via the urea cycle. Polyamine synthesis is highly regulated by rapid turnover of the synthetic enzymes, by feedback inhibition, and by endogenous inhibitors of ODC such as antizyme. Indeed, polyamine homeostasis overall relies on a balance between polyamine biosynthesis and catabolic pathways that degrade and provide a recycling mechanism for polyamine pools. Therefore, targeting the enzymes that synthesize and deplete polyamines is a potential way to influence β cell polyamine concentrations.

The rate-limiting enzyme in intracellular polyamine biosynthesis is ornithine decarboxylase (ODC). ODC converts ornithine to putrescine (1), which is then converted by spermidine synthase into spermidine (2). The process of spermidine synthesis also requires the action of another enzyme, S-adenosylmethionine decarboxylase (AMD). AMD decarboxylates S-adenosylmethionine (SAM) yielding a decarboxylated SAM (dcSAM), which donates a propyl amine moiety to form spermidine. Spermidine in turn is converted to spermine (3) by spermine synthase (using another dcSAM). Since ODC is highly regulated, putrescine is normally present in cells at low levels. This control is necessary in part because putrescine binds an allosteric AMD site and increases AMD activity eight-fold. When putrescine is present in cells in excess, the increase in AMD activity depletes SAM concentrations, and suppresses important methylation events. In this regard, there are direct links between intracellular polyamine levels and gene expression.

Specifically, a pair of enzymes, spermidine/spermine-N1-acetyltransferase (SSAT) and polyamine oxidase (PAO) work stepwise to convert spermine to spermidine and spermidine to putrescine. While spermine oxidase has been shown to directly convert spermine to spermidine (Hong et al., 2010), PAO activity results in the production of amido-aldehydes, and H₂O₂ (FIG. 1). The amido-aldehydes then form malondialdehyde and acrolein. Moreover, the action of the related diamine oxidase (DAO) generates ammonia and hydrogen peroxide (Seiler, 2004). Therefore, several of the products of these amine catabolic pathways induce oxidative stress within cells. In summary, perturbations in intracellular polyamine levels can lead to significant oxidative stress in mammalian cells.

As noted above, PAO activity results in the generation of reactive oxygen species (hydrogen peroxide) which increase oxidative stress. The accumulation of polyamines appears to be detrimental to the health and function of β cells, especially in the setting of autoimmunity and β cell stress (Brook, 2012; Maier et al., 2010; Maier et al., 2010; Templin et al., 2011; Bjelakovic et al., 2010; Nishiki et al., 2013). Polyamines also may play a role in the development of clinical complications. Children with T1D have been shown to have increased PAO activity (Bjelakovic et al., 2010). Adults with two other autoimmune diseases, Sjogren's syndrome and rheumatoid arthritis, have increased polyamine recycling back through the SSAT/PAO pathways, increasing intracellular putrescine (Higashi et al., 2010; Furumitsu et al., 2000; Furumitsu et al., 1993).

Dietary polyamine intake also influences the total body polyamine load (Bardocz et al., 1995). Indeed, putrescine, spermidine, and spermine are each found in foods commonly consumed in Western diets (Zoumas-Morse et al., 2007). Polyamines are also made by intestinal bacteria (Milovic, 2001). Enteral polyamines from foods and gut flora are quickly absorbed from the intestine and distributed throughout the body. Long-term polyamine-rich food consumption increases steady-state blood polyamine concentrations (Soda et al., 2009).

Several studies have implicated polyamines in carcinogenesis because they promote the production of cell cycle proteins and stabilize nucleic acids during cell replication. As such, depletion of cellular polyamines using eflornithine (a clinically approved irreversible inhibitor of ODC) has remained an attractive approach to diminish replication and enhance apoptosis rates in a variety of cancers (Jeter et al., 2012).

In contrast to cancer cells, islet β cells have remarkably low replicative capacity and may be less dependent upon polyamines for replication. Depletion of cellular polyamines with DFMO appears to have little effect on islet replication in vitro, and paradoxically may promote replication in islets from lean mice (Sjoholm et al., 2001). Studies of Berggren and colleagues in the early 1990s suggested that eflornithine improves insulin content and secretion in RINm5F insulinoma cells, the latter through effects of polyamines on voltage-dependent Ca2+ channels (Sjoholm et al., 1993). Based on the published literature and other own studies, the inventors hypothesized that polyamines contribute to β cell dysfunction/death in T1D in at least three ways. First, high levels of intracellular polyamines are associated with low levels of the caveolar protein Cav-1 (Roy et al., 2008; Belting et al., 2003; Belting et al., 1999; Welch et al., 2008), a protein required for stabilization of caveolae and normal glucose-responsive insulin release (Nevins et al., 2006). Second, polyamine degradation via polyamine oxidase results in the formation of reactive oxygen species, aminoaldehydes, which are then spontaneously converted to malondialdehyde and acrolein, causing oxidative and ER stress (Brooks, 2012; Maier et al., 2010; Maier et al., 2010; Templin et al., 2011; Bjelakovic et al., 2010; Nishiki et al., 2013). Third, the polyamine spermidine is a necessary substrate for the formation of the active, hypusine form of the pro-inflammatory translation elongation factor eIF5A (eIF5AHyp) (Park et al., 2010). Whereas data on the former two mechanisms in β cells are limited, data on the third are much more developed. eIF5AHyp in the β cell participates in the translational elongation of a subset of mRNAs (most notably inducible nitric oxide synthase), that are responsive to pro-inflammatory cytokines (Jeter et al., 2012; Maier et al., 2010; Templin et al., 2011; Nishiki et al., 2013). In the setting of β cell ER stress, eIF5AHyp localizes to the ER, where it appears to promote the translational elongation of the crucial ER stress mRNA Chop (Robbins et al., 2010).

Without being bound by theory, the effect of polyamines on the pathogenesis of T1D may be mediated by influences on epigenetic chromatin methylation that plays a critical role in the regulation of gene expression (Brooks, 2012). Many autoimmune diseases, including T1D have been associated with abnormalities in methylation (Renaudineau et al., 2011). As mentioned above, SAM is required for polyamine synthesis and provides methyl groups for cellular methylation events (Brooks, 2012). Although SAM is usually abundant in cells, increased throughput in the polyamine pathway can deplete SAM, disrupting chromatin methylation, leading to abnormal expression of otherwise sequestered genes that incite the autoimmune process in T1D.

Excess polyamines appear to play a role in accelerating β cell ER stress and apoptosis (Packham et al., 1995). Specifically, spermidine is necessary for the production of the hypusine modification of the translational factor eIF5A. Hypusinated eIF5A (eIF5A-Hyp) is also central to the β cell apoptotic response to inflammation. In the setting of β cell ER stress, eIF5A-Hyp localizes to the ER, where it appears to promote the translational elongation of the crucial ER stress mRNA Chop (Robbin et al., 2010). One means of measuring ER stress clinically is to examine the relative amounts of circulating proinsulin and C-peptide. The inventors have identified a key pattern of dysfunctional insulin secretion in pre-diabetic NOD mice characterized by increased serum levels of proinsulin relative to serum levels of the mature and fully-processed insulin molecule (assessed by measuring C-peptide), a finding that suggests alterations in protein folding and dysfunction at the level of the ER (Tersey et al., 2012). The inventors have validated pro-insulin to c-peptide in a pilot study of 20 new onset persons compared to matched controls, finding that pro-insulin to c-peptide ratios were elevated at diagnosis and that these elevations were sustained 8 weeks later despite improved glycemic control (Watkins et al., 2013).

### I. TYPE 1 DIABETES

Type 1 diabetes mellitus (Type 1 diabetes), also called insulin dependent diabetes mellitus or juvenile diabetes, is a form of diabetes mellitus that results from autoimmune destruction of insulin-producing beta cells of the pancreas. The subsequent lack of insulin leads to increased blood glucose concentrations and increased urinary glucose excretion. The classical symptoms are polyuria, polydipsia, polyphagia, and weight loss. Type 1 diabetes may be fatal unless treated with insulin.

Type 1 diabetes is a condition in which a subject has, in the presence of autoimmunity towards the pancreatic beta-cell or insulin, a fasting blood glucose or serum glucose concentration greater than 125 mg/dL (6.94 mmol/L). If a glucose tolerance test is carried out, the blood sugar level of a diabetic will be in excess of 200 mg of glucose per dL (11.1 mmol/l) of plasma 2 hours after 75 g of glucose have been taken on an empty stomach, in the presence of autoimmunity towards the pancreatic beta cell or insulin. In a glucose tolerance test, 75 g of glucose are administered orally to the patient being tested after 10-12 hours of fasting and the blood sugar level is recorded immediately before taking the glucose and 1 and 2 hours after taking it. The presence of autoimmunity towards the pancreatic beta-cell may be observed by detection of circulating islet cell autoantibodies [“type 1A diabetes mellitus”], i.e., at least one of: GAD65 [glutamic acid decarboxylase-65], ICA [islet-cell cytoplasm], IA-2 [intracytoplasmatic domain of the tyrosine phosphatase-like protein IA-2], ZnT8 [zinc-transporter-8] or anti-insulin; or other signs of autoimmunity without the presence of typical circulating autoantibodies [type 1B diabetes], i.e. as detected through pancreatic biopsy or imaging). Typically, a genetic predisposition is present (e.g. HLA, INS VNTR and PTPN22), but this is not always the case.

Large randomized studies have established that intensive and tight glycemic control during early (newly diagnoses to 5 years) stage diabetes has enduring beneficial effects and reduces the risk of diabetic complications, both micro- and macrovascular. However, many patients with diabetes still develop diabetic complications despite receiving intensified glycemic control.

Standard therapy of type 1 diabetes is insulin treatment. Therapies for type 1 diabetes are for example described in WO 2012/062698, which is incorporated by reference herein in its entirety.

C-peptide originates from proinsulin and is produced in the body along with insulin. It is an accepted biomarker for proof of beta-cell preservation. Other biomarkers of β cell stress include unmethylated DNA and HSP90.

In one embodiment, diabetes patients within the meaning of this invention may include patients who have not previously been treated with an antidiabetic drug (drug-naïve patients). Thus, in an embodiment, the therapies described herein may be used in naïve patients.

A further embodiment of diabetic patients within the meaning of this invention refers to patient having type 1 diabetes with or at risk of developing micro- or macrovascular diabetic complications, such as e.g. retinal complications (e.g., diabetic retinopathy), macrovascular complications (e.g., myocardial infarction, coronary artery disease, ischemic or hemorrhagic stroke, and/or peripheral occlusive arterial disease), or cardiovascular disease or events.

Diabetic nephropathy is a complication of diabetes that evolves early, typically before clinical diagnosis of diabetes is made. The earliest clinical evidence of nephropathy is the appearance of low but abnormal levels (>30 mg/day or 20 μg/min) of albumin in the urine (microalbuminuria), followed by albuminuria (>300 mg/24 h or ˜200 μg/min) that develops over a period of 10-15 years. In patients with type 1 diabetes, diabetic hypertension typically becomes manifest early on, by the time that patients develop microalbuminuria. Once overt nephropathy occurs, the glomerular filtration rate (GFR) falls over several years resulting in End Stage Renal Disease (ESRD) in 50% of patient with type 1 diabetes within 10 years and in >75% of patient with type 1 diabetes by 20 years of onset of overt nephropathy. Albuminuria (i.e., proteinuria) is a marker of greatly increased cardiovascular morbidity and mortality for patients with either type 1 or type 2 diabetes.

The effect of diabetes on the eye is called diabetic retinopathy and involves changes to the circulatory system of the retina. The earliest phase of the disease is known as background diabetic retinopathy wherein the arteries in the retina become weakened and leak, forming small, dot-like hemorrhages. These leaking vessels often lead to swelling or edema in the retina and decreased vision. The next stage is proliferative diabetic retinopathy, in which circulation problems cause areas of the retina to become oxygen-deprived or ischemic. New vessels develop as the circulatory system attempts to maintain adequate oxygen levels within the retina Unfortunately, these new vessels hemorrhage easily. In the later phases of the disease, continued abnormal vessel growth and scar tissue may cause serious problems such as retinal detachment and glaucoma. First agents are used to treat, prevent, reduce or ameliorate diabetic retinopathy. The agents can be administered by the methods described below, including by topical administration to the eye. The agents can also be administered by intravitreal implant.

Diabetic neuropathies are a family of nerve disorders caused by diabetes which can be very painful. Pain derived from a diabetic sensory neuropathy is the most common form of diabetic neuropathy. Predominant pain may be combined with temperature and tactile loss. The pain is usually aching, prickling, or burning in quality with superimposed stabs, and often most troublesome at night. The pain is felt predominantly in the lower limbs, however, with occurrence also at the upper limbs and trunk.

Diabetic cardiomyopathy is a disease of the heart muscle (myocardium).

Diabetic cardiomyopathy clinically expresses itself as congestive heart failure (CHF) and left ventricular hypertrophy. Diabetic cardiomyopathy is also associated with increased morbidity and mortality. Pathologically, diabetic cardiomyopathy is characterized by myocellular hypertrophy, interstitial fibrosis, increased myocardial lipid deposition, and varying degrees of small vessel disease. Diabetic cardiomyopathy differs from ischemic cardiomyopathy because the diseased myocardium and resultant CHF can occur in the absence of frank coronary atherosclerosis or luminal narrowing. This suggests that the primary metabolic defects related to hyperglycemia that exist in the myocardial tissue and/or in the coronary microcirculation itself are responsible for the diseased state and loss of myocardial function in diabetics. Co-existent hypertension, microvascular complications, impaired fibrinolysis, atherosclerotic cardiovascular disease, and/or myocardial ischemia, which frequently occur in diabetic patients, compound the severity of the underlying diabetic cardiomyopathy. These co-morbidities can lower the threshold for decompensated heart failure, pulmonary edema, and arrhythmias, which can result in the death of the patient. Diabetic cardiomyopathy is associated with mechanical dysfunction of the heart. The hypertrophied fibrotic myocardium has reduced compliance, leading to diastolic dysfunction and an elevated left ventricular filling pressure. Progression of the cardiomyopathic process may ultimately result in impairments in myocardial contraction and systolic dysfunction. A reduced stroke volume, low ejection fraction, and impaired cardiac reserve will cause a further rise in left ventricular filling pressures. This may result in fulminant heart failure.

### II. EFLORNITHINE

The term “eflornithine” when used by itself and free of context refers to 2,5-diamino-2-(difluoromethyl) pentanoic acid in any of its forms, including non-salt and salt forms (e.g., eflornithine HCl), anhydrous and hydrate forms of non-salt and salt forms (e.g., eflornithine hydrochloride monohydrate), solvates of non-salt and salts forms, its enantiomers (R and S forms, which may also by identified as d and/forms), and mixtures of these enantiomers (e.g., racemic mixture). By “substantially optically pure preparation” is meant a preparation of a first enantiomer which contains about 5% wt. or less of the opposite enantiomer. Specific forms of eflornithine include eflornithine hydrochloride monohydrate (i.e., CAS ID: 96020-91-6; MW: 236.65), eflornithine hydrochloride (i.e., CAS ID: 68278-23-9; MW: 218.63), and anhydrous free base eflornithine (i.e., CAS ID: 70052-12-9; MW: 182.17). Where necessary, the specific form of eflornithine has been further specified. In some embodiments, the eflornithine of the present disclosure is eflornithine hydrochloride monohydrate (i.e., CAS ID: 96020-91-6). The terms “eflornithine” and “DFMO” are used interchangeably herein. DFMO is an abbreviation for difluoromethylornithine. Other synonyms of eflornithine and DFMO include: α-difluoromethylornithine, 2-(difluoromethyl)-DL-ornithine, 2-(difluoromethyl)-dl-ornithine, 2-(Difluoromethyl) ornithine, DL-α-difluoromethylornithine, N-Difluoromethylornithine, αδ-diamino-α-(difluoromethyl) valeric acid, and 2,5-diamino-2-(difluoromethyl) pentanoic acid.

Eflornithine is an enzyme-activated, irreversible inhibitor of ornithine decarboxylase (ODC), the first and the rate limiting enzyme of the polyamine biosynthetic pathway (Meyskens & Gerner, 1999). Eflornithine is well-tolerated in animals and humans and has been used clinically for over 40 years. Eflornithine was used in several studies (NMTRC002, NMTRC003, and NANT 2012-01) of children with neuroblastoma and has been well-tolerated by children.

Eflornithine has been shown to decrease APC-dependent intestinal tumorigenesis in mice (Erdman et al., 1999). Oral eflornithine administered daily to humans inhibits ODC enzyme activity and polyamine contents in a number of epithelial tissues (Love et al., 1993; Gerner et al., 1994; Meyskens et al., 1994; Meyskens et al., 1998; Simoneau et al., 2001; Simoneau et al., 2008). Eflornithine in combination with the non-steroidal anti-inflammatory drug (NSAID) sulindac, has been reported to markedly lower the adenoma occurrence rate among individuals with colonic adenomas when compared to placebos in a randomized clinical trial (Meyskens et al., 2008).

Eflornithine is relatively non-toxic at low doses of 0.4 g/m²/day to humans while producing inhibition of putrescine synthesis. Side effects observed with eflornithine include effects on hearing at high doses of 4 g/m²/day that resolve when it is discontinued. These effects on hearing are not observed at lower doses of 0.4 g/m²/day when administered for up to one year (Meyskens et al., 1994). In addition, a few cases of dizziness/vertigo are seen that resolve when the drug is stopped. Thrombocytopenia has been reported predominantly in studies using high “therapeutic” doses of eflornithine (>1.0 g/m²/day) and primarily in cancer patients who had previously undergone chemotherapy or patients with compromised bone marrow. Although the toxicity associated with eflornithine therapy are not, in general, as severe as other types of chemotherapy, in limited clinical trials it has been found to promote a dose-related thrombocytopenia. Moreover, studies in rats have shown that continuous infusion of eflornithine for 12 days significantly reduces platelet counts compared with controls. Other investigations have made similar observations in which thrombocytopenia is the major toxicity of continuous i.v. eflornithine therapy. These findings suggest that eflornithine may significantly inhibit ODC activity of the bone marrow precursors of megakaryocytes. Eflornithine may inhibit proliferative repair processes, such as epithelial wound healing. A phase III clinical trial assessed the recurrence of adenomatous polyps after treatment for 36 months with eflornithine plus sulindac or matched placebos.

Eflornithine is known to deplete T cells in mice (Bowlin et al., 1986). Administration of eflornithine to a variety of mouse models (including the NOD mouse) preserves β cell function and delays diabetes onset (Tersey et al., 2014). Inhibition of ODC using eflornithine in vivo has also been shown to reduce polyamine concentrations in a variety of tissues in both humans and mice (Jeter et al., 2012).

A specific association between the increase in polyamine synthesis and some primary and or secondary events involved in eukaryotic cellular growth and differentiation processes has been clearly identified. Polyamine biosynthesis has been associated with cell transformation, chemical-induced carcinogenesis, and experimental tumor cell proliferation. Experimental data indicate that inhibition of polyamine biosynthesis results in either a stimulatory or inhibitory effect on cellular differentiation depending on the model studied. Accordingly, eflornithine treatment has resulted in opposite effects on cell differentiation in a variety of models.

### III. TREATMENT OF PATIENTS

In some embodiments, the treatment methods may be supplemented with diagnostic methods to improve the efficacy and/or minimize the toxicity of eflornithine. Such methods are described, for example, in U.S. Pat. Nos. 8,329,636 and 9,121,852, U.S. Patent Publications US2013/0217743 and US2015/0301060, and PCT Patent Publications WO2014/070767 and WO2015/195120, which are all incorporated herein by reference.

In some embodiments, compositions and formulations of the present disclosure may be administered to a subject with a genotype at position +316 (rs2302615) of at least one allele of the ODC1 gene promoter is G. In some embodiments, the genotype at position +316 of both alleles of the patient's ODC1 gene promoters may be GG. In some embodiments, the genotype at position +316 (rs2302615) of both alleles of the patient's ODC1 gene promoters may be GA. ODC1 A allele carriers at position +316 (rs2302615) differ in response to prolonged exposure with eflornithine and sulindac compared to GG genotype patients, with A allele carriers experiencing potential for elevated risk of developing ototoxicity, especially among the AA homozygotes.

In some embodiments, a patient with type 1 diabetes is treated by methods that comprise: (a) obtaining results from a test that determines the patient's genotype at position +316 (rs2302615) of at least one ODC1 promoter gene allele; and (b) if the results indicate that the patient's genotype at position +316 (rs2302615) of at least one allele of the ODC1 promoter gene is G, then administering to the patient a composition comprising eflornithine. In some embodiments, diabetic complications are prevented, slowed, delayed, or treated in a patient having type 1 diabetes by methods that comprise: (a) obtaining results from a test that determines the patient's genotype at position +316 (rs2302615) of at least one ODC1 promoter gene allele; and (b) if the results indicate that the patient's genotype at position +316 (rs2302615) of at least one allele of the ODC1 promoter gene is G, then administering to the patient a composition comprising eflornithine. See U.S. Pat. No. 8,329,636, which is incorporated herein by reference.

In some embodiments, a patient with type 1 diabetes is treated by methods that comprise administering to the patients a composition comprising eflornithine, wherein the patient has been determined to have a dietary polyamine intake, and/or tissue polyamine level, and/or tissue polyamine flux that is not high. In some of these embodiments, the dietary polyamine intake that is not high is 300 μM polyamine per day or lower. See U.S. Pat. No. 10,151,756, which is incorporated herein by reference.

In some embodiments, a patient with type 1 diabetes is treated by methods that comprise: (a) obtaining results from a test that determines the patient's genotype at position +263 (rs2302616) of at least one ODC1 allele; and (b) if the results indicate that the patient's genotype at position +263 (rs2302616) of at least one allele of the ODC1 gene is T, then administering to the patient a composition comprising eflornithine. In some of these embodiments, the test may determine the nucleotide base at position +263 (rs2302616) of one allele of the ODC1 gene in the patient. In some embodiments, the test may determine the nucleotide bases at position +263 (rs2302616) of both alleles of the ODC1 gene in the patient. In some embodiments, the results may indicate that the patient's genotype at position +263 (rs2302616) of both alleles of the ODC1 gene is TT. In some embodiments, the results may indicate that the patient's genotype at position +263 (rs2302616) of both alleles of the ODC1 gene is TG. In some of these embodiments, the method may further comprise obtaining results from a test that determines the patient's genotype at position +316 (rs2302615) of at least one ODC1 allele and only administering to the patient of the composition provided herein if the results indicate that the patient's genotype at position +316 (rs2302615) of at least one allele of the ODC1 gene is G. In another aspect, diabetic complications are prevented, slowed, delayed, or treated in a patient having type 1 diabetes by methods that comprise: (a) obtaining results from a test that determines the patient's genotype at position +263 (rs2302616) of at least one ODC1 allele; and (b) if the results indicate that the patient's genotype at position +263 (rs2302616) of at least one allele of the ODC1 gene is T, then administering to the patient a composition comprising eflornithine. See PCT Patent Publication WO2015/195120, which is incorporated herein by reference.

In variations on any of the above embodiments, the patient is human.

In some embodiments, the eflornithine may be administered on a routine schedule. As used herein, a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the eflornithine may be taken orally and that the timing of which is or is not dependent upon food intake. Thus, for example, the eflornithine can be taken every morning and/or every evening, regardless of when the subject has eaten or will eat.

In some embodiments, the eflornithine is administered in combination with at least a second agent. The at least second therapy may precede or follow treatment with eflornithine by intervals ranging from minutes to months. In some aspects, one would ensure that a significant period of time did not expire between the time of each delivery, such that the combination of agents would still be able to exert an advantageously combined effect. In such instances, one would typically administer the combination of eflornithine and the at least second therapeutic agent within about 12-24 hours of each other and, more preferably, within about 6-12 hours of each other. In some aspects, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, such as where “A” represents eflornithine and “B” represents the at least second agent, non-limiting examples of which are described below:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that agents that modulate the polyamine pathway may be used in conjunction with the treatments of the current invention. For example, non-steroidal anti-inflammatory drugs (NSAIDs), polyamine transporter inhibitors, eIF-5A antagonists, and structural polyamine analogs (SPA) may be used.

### A. NSAIDs

NSAIDs are anti-inflammatory agents that are not steroids. In addition to anti-inflammatory actions, they have analgesic, antipyretic, and platelet-inhibitory actions. They are used primarily in the treatment of chronic arthritic conditions and certain soft tissue disorders associated with pain and inflammation. They act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms may contribute to their anti-inflammatory effects. Certain NSAIDs also may inhibit lipoxygenase enzymes or phospholipase C or may modulate T-cell function. Examples of NSAIDS that may be used include, but are not limited to, aspirin, ibuprofen, naproxen, fenoprofen, ketoprofen, flurbiprofen, oxaprozin, indomethacin, etodolac, diclofenac, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, and etoricoxib.

Sulindac is a non-steroidal anti-inflammatory drug (NSAID) that exhibits anti-inflammatory, analgesic and antipyretic activities in animal models. Sulindac sulfone induces peroxisome proliferator-activated receptor-γ (PPAR), which binds to PPAR response elements for the spermidine/spermine acetyltransferase (SAT1) gene. Activation of this gene promotes the export of polyamines. This mechanism is complementary to the mechanism of eflornithine in reducing polyamine levels. Experimental findings in human cell and mouse models indicate that sulindac and other NSAIDS activate polyamine catabolismhus, NSAIDs complement inhibitors of polyamine synthesis, like eflornithine, to reduce tissue polyamines.

Sulindac is a nonsteroidal, anti-inflammatory indene derivative with the following chemical designation; (Z)-5-fluoro-2-methyl-1-((4-(methylsulfinyl)phenyl)methylene)-1H-indene-3-acetic acid. Without being bound by theory, the sulfinyl moiety is converted in vivo by reversible reduction to a sulfide metabolite and by irreversible oxidation to a sulfone metabolite (exisulind).

Sulindac is available, for example, as 150 mg and 200 mg tablets. The most common dosage for adults is 150 to 200 mg twice a day, with a maximal daily dose of 400 mg. After oral administration, about 90% of the drug is absorbed. Peak plasma levels are achieved in about 2 hours in fasting patients and 3 to 4 hours when administered with food. The mean half-life of sulindac is 7.8 hours: the mean half-life of the sulfide metabolite is 16.4 hours. U.S. Pat. Nos. 3,647,858 and 3,654,349 cover preparations of sulindac, both are incorporate by reference herein in their entireties.

Sulindac is indicated for the acute and long-term relief of signs and symptoms of osteoarthritis, rheumatoid arthritis, ankylosing spondylitis, acute gout, and acute painful shoulder. The analgesic and antiinflammatory effects exerted by sulindac (400 mg per day) are comparable to those achieved by aspirin (4 g per day), ibuprofen (1200 mg per day), indomethacin (125 mg per day), and phenylbutazone (400 to 600 mg per day). Side effects of sulindac include mild gastrointestinal effects in nearly 20% of patients, with abdominal pain and nausea being the most frequent complaints. CNS side effects are seen in up to 10% of patients, with drowsiness, headache, and nervousness being those most frequently reported. Skin rash and pruritus occur in 5% of patients. Chronic treatment with sulindac can lead to serious gastrointestinal toxicity such as bleeding, ulceration, and perforation.

A combination therapy of DFMO and sulindac was shown to be effective in reducing adenomas in these mice. See U.S. Pat. No. 6,258,845, which is incorporated herein by reference in its entirety.

Celecoxib is a non-steroidal anti-inflammatory agent that is well established in the treatment of osteoarthritis, rheumatoid arthritis, acute pain, ankylosing spondylitis, and to reduce the number of colon and rectal polyps in patients with FAP with the following chemical designation: 4-[5-(4-Methylphenyl)-3-(trifluoromethyl) pyrazol-1-yl]benzenesulfonamide. Celecoxib is a selective COX-2 inhibitor. Side effects of celecoxib include a 30% increase in rates of heart and blood vessel disease. Additionally, the risk of gastrointestinal side effects is greater than 80%.

Combinations of various NSAIDs are also used for various purposes. By using lower doses of two or more NSAIDs, it is possible to reduce the side effects or toxicities associated with higher doses of individual NSAIDs. For example, in some embodiments, sulindac may be used together with celecoxib. In some embodiments, the one or both of the NSAIDS are selective COX-2 inhibitors.

In some aspects, the present methods comprise administering a fixed dose combination of a pharmaceutically effective amount of eflornithine and a pharmaceutically effective amount of a nonsteroidal anti-inflammatory drug (NSAID) or a metabolite thereof. In some embodiments, the fixed dose combination is a pharmaceutically effective amount of eflornithine and a pharmaceutically effective amount of sulindac. Examples of such fixed dose combination are provided in International PCT Publication Number WO 2017/075576, which is incorporated by reference herein in its entirety. In some aspects, the present methods comprise separately administering a pharmaceutically effective amount of eflornithine and a pharmaceutically effective amount of a nonsteroidal anti-inflammatory drug (NSAID) or a metabolite thereof.

### B. Polyamine Transporter Inhibitors

Inhibitors of the polyamine transport include, but are not limited to, 4-bis(3-aminopropyl)-piperazine (BAP) and compounds disclosed in U.S. Patent Publn. No. 2011/0256161 (e.g., AMXT1501); U.S. Patent Publn. No. 2012/0172449; PCT Publn. No. WO 1999/054283; U.S. Pat. Nos. 6,083,496; and 5,456,908.

### C. Structural Polyamine Analogs (SPA)

SPA decrease polyamines by negatively regulating the polyamine biosynthetic enzymes and positively regulating polyamine catabolic enzymes. Some have already been developed and are in clinical trials. A phase II study of the SPA DENSPM found it to be well-tolerated.

### D. Enzyme Inhibitors

The second agent could be another enzyme inhibitor targeting ODC, AMD or PAO. Phase I and II trials of these agents are ongoing, including the AMD inhibitor SAM4861.

### IV. PHARMACEUTICAL FORMULATIONS AND ROUTES OF ADMINISTRATION

In some embodiments, the eflornithine is eflornithine hydrochloride monohydrate. In some embodiments, the eflornithine is eflornithine hydrochloride monohydrate racemate. In some embodiments, the eflornithine hydrochloride monohydrate is a racemic mixture of its two enantiomers.

In some embodiments, the eflornithine is present in an amount of about 10 to about 1000 mg. In some embodiments, the eflornithine is present in an amount of about 250 to about 500 mg. In some embodiments, the eflornithine is present in an amount of about 300 to about 450 mg. In some embodiments, the eflornithine is present in an amount of about 350 to about 400 mg. In some embodiments, the eflornithine is present in an amount of about 35 to about 60 weight percent. In some embodiments, the eflornithine is present in an amount of about 40 to about 55 weight percent. In some embodiments, the eflornithine is present in an amount of about 50 to about 55 weight percent. In some embodiments, the eflornithine is present in an amount of about 52 to about 54 weight percent. In some embodiments, the amount of eflornithine hydrochloride monohydrate racemate is from 52 to 54 weight percent. In some embodiments, the eflornithine is present in an amount of about 375 mg. In some embodiments, the amount of eflornithine hydrochloride monohydrate racemate is 375 mg.

In some embodiments, when sulindac is administered as a fixed dose combination with eflornithine, the sulindac is present in an amount from about 10 to about 1500 mg. In some embodiments, the sulindac is present in an amount of about 50 to about 100 mg. In some embodiments, the sulindac is present in an amount of about 70 to about 80 mg. In some embodiments, the sulindac is present in an amount of about 75 mg. In some embodiments, the amount of sulindac is 75 mg. In some embodiments, the sulindac is present in an amount of about 5 to about 20 weight percent. In some embodiments, the sulindac is present in an amount of about 8 to about 15 weight percent. In some embodiments, the sulindac is present in an amount of about 10 to about 12 weight percent. In some embodiments, the amount of sulindac is from 10 to 11 weight percent.

In some embodiments, the eflornithine is present in an amount of about 375 mg and the sulindac is present in an amount of about 75 mg.

In some embodiments, the formulation further comprises an excipient. In some embodiments, the excipient is starch, colloidal silicon dioxide, or silicified microcrystalline cellulose. In some embodiments, the excipient is colloidal silicon dioxide. In some embodiments, the formulation further comprises a second excipient. In some embodiments, the second excipient is silicified microcrystalline cellulose.

In some embodiments, the formulation further comprises a lubricant. In some embodiments, the lubricant is magnesium stearate, calcium stearate, sodium stearate, glyceryl monostearate, aluminum stearate, polyethylene glycol, boric acid or sodium benzoate. In some embodiments, the lubricant is magnesium stearate. In some embodiments, magnesium stearate is present in an amount of about 0.25 to about 2 weight percent. In some embodiments, the amount of magnesium stearate is from about 0.75 to about 2 weight percent. In some embodiments, the amount of magnesium stearate is from about 1 to about 1.5 weight percent. In some embodiments, the amount of magnesium stearate is about 1.1 weight percent. In some embodiments, magnesium stearate is present in an amount of about 1.5 weight percent.

In some embodiments, the compositions are in the form of a capsule, tablet, mini tablet, granule, or pellet. In some embodiments, the composition is in the form of a tablet, for example, a monolayer tablet.

In some embodiments, the weight of the tablet is from about 650 mg to about 1,000 mg. In some embodiments, the weight of the tablet is from about 675 mg to about 725 mg. In some embodiments, the weight of the tablet is about 700 mg.

In some embodiments, the tablet further comprises a coating. In some embodiments, the coating is a modified release coating or an enteric coating. In some embodiments, the coating is a pH-responsive coating. In some embodiments, the coating comprises cellulose acetate phthalate (CAP), cellulose acetate trimelletate (CAT), poly(vinyl acetate) phthalate (PVAP), hydroxypropylmethylcellulose phthalate (HP), poly(methacrylate ethylacrylate) (1:1) copolymer (MA-EA), poly(methacrylate methylmethacrylate) (1:1) copolymer (MA MMA), poly(methacrylate methylmethacrylate) (1:2) copolymer, or hydroxypropylmethylcellulose acetate succinate (HPMCAS). In some embodiments, the coating masks the taste of eflornithine. In some embodiments, the coating comprises hydroxypropyl methylcellulose, titanium dioxide, polyethylene glycol, and iron oxide yellow.

In some embodiments, the amount of coating is from about 1 to about 5 weight percent. In some embodiments, the amount of coating is from about 2 to about 4 weight percent. In some embodiments, the amount of coating is about 3 weight percent. In some embodiments, the amount of coating is from about 5 mg to about 30 mg. In some embodiments, the amount of coating is from about 15 mg to about 25 mg. In some embodiments, the amount of coating is about 21 mg.

In some embodiments, the weight of the tablet comprising a coating is from about 675 mg to about 750 mg. In some embodiments, the weight of the tablet comprising a coating is from about 700 mg to about 725 mg. In some embodiments, the weight of the tablet comprising a coating is about 721 mg.

In some embodiments, the pharmaceutical compositions and formulations of the present invention are for enteral, such as oral, and also rectal or parenteral, with the compositions comprising the pharmacologically active compounds either alone or together with pharmaceutical auxiliary substances (excipients). Pharmaceutical preparations for enteral or parenteral administration are, for example, in unit dose forms, such as coated tablets, tablets, capsules or suppositories and also ampoules. These are prepared in a manner, which is known per se, for example using conventional mixing, granulation, coating, solubilizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances. In a preferred embodiment, a mixture of active ingredients and excipients are formulated into a tablet form. Appropriate coatings may be applied to increase palatability or delay absorption. For example, a coating may be applied to a tablet to mask the disagreeable taste of the active compound, such as eflornithine, or to sustain and/or to delay the release of the active molecules to a certain area in the gastrointestinal tract.

The therapeutic compounds can be orally administered, for example, with an inert diluent or an assimilable edible carrier. The therapeutic compounds and other ingredients may also be enclosed in a hard or soft shell gelatin capsule, compressed into tablets, or incorporated directly into the subject's diet. For oral therapeutic administration, the therapeutic compounds may be incorporated with excipients and used in the form of ingestible tablets, buccal tablets, troches, capsules, elixirs, suspensions, syrups, or wafers.

In certain embodiments, the tablets and/or capsules provided herein comprise the active ingredients and powdered carriers, such as lactose, starch, cellulose derivatives, magnesium stearate, and stearic acid. Similar diluents can be used to make compressed tablets. In other embodiments, tablets and capsules can be manufactured for immediate or modified release. In some embodiments, the tablet and/or capsule is manufactured as a sustained release product to provide for continuous release of medication over a period of hours. In some embodiments, the compressed tablet is sugar-coated and/or film-coated to mask unpleasant taste and/or protect the tablet from the atmosphere. In some embodiments, the tablet is enteric coated for selective disintegration in the gastrointestinal tract.

In some embodiments, the tablet or capsule is able to disintegrate or dissolve to liberate multiparticulates comprising particles of different populations of a first component and a second component, e.g. modified release coated multiparticles. In some of these embodiments, the tablet or capsule may disintegrate or dissolve in the mouth, stomach, small intestine, terminal ileum, or colon. In some of these embodiments, the tablet or capsule may release the multiparticulates with modified release properties.

In some embodiments, the present invention encompasses methods of administering a pharmaceutical oral fixed dose combination in the form of a multilayer tablet. A multilayer tablet has at least two layers (bilayer tablet) or can have three, four, five or more layers. In some embodiments, each of the layers contains not more than one of the active pharmaceutical ingredients (APIs). For example, in some embodiments, the tablet has two layers, with one of the APIs in each of the two layers. In some embodiments, in addition to these two layers, the tablet contains further layers containing only carrier and which may function, e.g., as separation layer(s) or outer coating layer(s). In some embodiments, if more than two layers are present, the components may be present in more than one layer as long as they are not present together in the same layer. In certain embodiments, a monolayer tablet is preferred but all information detailed below is equally applicable to multilayer tablets.

In some embodiments, the compositions further comprise a pharmaceutically acceptable excipient. In some of these embodiments, the pharmaceutically acceptable excipient may include a pharmaceutically acceptable diluent, a pharmaceutically acceptable disintegrant, a pharmaceutically acceptable binder, a pharmaceutically acceptable stabilizer, a pharmaceutically acceptable lubricant, a pharmaceutically acceptable pigment, or pharmaceutically acceptable glider. In a fixed dose combination formulation of the present invention, an active ingredient may be mixed at a weight ratio of 1:0.25 to 1:20 with a pharmaceutically acceptable excipient.

Diluents that can be used in pharmaceutical formulations of the present invention include, but are not limited to, microcrystalline cellulose (“MCC”), silicified MCC (e.g. PROSOLV™ HD 90), microfine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and any mixtures thereof. Preferably, the diluent is silicified MCC. The diluent may be used in an amount of from about 5 to about 95 weight percent based on the total weight of the formulation, and preferably in an amount of from about 25 to about 40 percent weight, such as in an amount of from about 30 to about 35 percent weight. In certain aspects, the diluent can be a soluble diluent. When the diluent is used, its ratio to the active ingredient in each discrete layer is very important. The term “soluble diluents” refers to a diluent which is dissolved in water, like lactose, Ludipress (BASF, a mixture of lactose, crospovidone and povidone (93:3.5:3.5, w/w (%))), mannitol and sorbitol.

Disintegrants are used to promote swelling and disintegration of the tablet after exposure to fluids in the oral cavity and/or gastrointestinal tract. Examples of disintegrants useful in the fixed dose combination formulation of the present invention include crospovidone, sodium starch glycolate, croscarmellose sodium, low-substituted hydroxypropylcellulose, starch, alginic acid or sodium salt thereof, and a mixture thereof. Other disintegrants that can be used in pharmaceutical formulations of the present invention include, but are not limited to, methylcelluloses, microcrystalline celluloses, carboxymethyl cellulose calcium, carboxymethyl cellulose sodium (e.g. AC-DI-SOL™, PRIMELLOSE™), povidones, guar gum, magnesium aluminum silicate, colloidal silicon dioxide (e.g. AEROSIL™, CARBOSIL™), polacrilin potassium, starch, pregelatinized starch, sodium starch glycolate (e.g. EXPLOTAB™), sodium alginate, and any mixtures thereof. Preferably, the disintegrant is colloidal silicon dioxide. The disintegrant may be used in an amount of about 0.1 to about 30 weight percent based on the total weight of the formulation, and preferably in an amount of about 0.2 to about 5 weight percent.

Compositions of the present invention may comprise lubricants. Sticking can occur when granules attach themselves to the faces of tablet press punches. Lubricants are used to promote flowability of powders, and to reduce friction between the tablet punch faces and the tablet punches and between the tablet surface and the die wall. For example, lubricants include magnesium stearate, calcium stearate, zinc stearate, stearic acid, sodium stearyl fumarate, polyethylene glycol, sodium lauryl sulphate, magnesium lauryl sulphate, and sodium benzoate. Preferably, the lubricant is magnesium stearate. In the present invention, lubricants preferably comprise 0.25 weight percent to 2 weight percent of the solid dosage form, and preferably in an amount of about 1.5 weight percent. In an exemplary formulation, the lubricant is magnesium stearate present in an amount of about 1.5 weight percent to prevent sticking.

Binders can be used in the pharmaceutical compositions of the present invention to help hold tablets together after compression. Examples of binders useful for the present invention are acacia, guar gum, alginic acid, carbomers (e.g. Carbopol™ products), dextrin, maltodextrin, methylcelluloses, ethylcelluloses, hydroxyethyl celluloses, hydroxypropyl celluloses (e.g. KLUCEL™), hydroxypropyl methylcelluloses (e.g. METHOCEL™), carboxymethylcellulose sodiums, liquid glucose, magnesium aluminum silicate, polymethacrylates, polyvinylpyrrolidones (e.g., povidone K-90 D, KOLLIDON™), copovidone (PLASDONE™), gelatin, starches, and any mixtures thereof. Preferably, the binder is starch. In the present invention, binders preferably comprise about 1 to about 15 weight percent of the solid dosage form. In other embodiments, the solid dosage form does not comprise a binder.

In certain embodiments, the stabilizer usable in the fixed dose combination formulation of the present invention may be an antioxidant. The use of an antioxidant enhances stability of the active ingredients against the undesirable reaction with other pharmaceutically acceptable additives and against modification by heat or moisture with time. For example, the antioxidant is ascorbic acid and its esters, butylated hydroxytoluene (BHT), butylated hydroxyanisole (BHA), α-tocopherol, cystein, citric acid, propyl gallate, sodium bisulfate, sodium pyrosulfite, ethylene diamine tetracetic acid (EDTA), and any mixtures thereof.

### V. DEFINITIONS

As used herein the specification, “a” or “an” may mean one or more. As used herein in the claim(s), when used in conjunction with the word “comprising,” the words “a” or “an” may mean one or more than one.

Throughout this application, the term “about” is used to indicate that a value includes the inherent variation of error for the device, that a value includes the inherent variation in the method being employed to determine the value, that a value includes the variation that exists among the study subjects, or a value that is within 10% of a stated value.

As used herein, the term “bioavailability” denotes the degree means to which a drug or other substance becomes available to the target tissue after administration. In the present context, the term “suitable bioavailability” is intended to mean that administration of a composition according to the invention will result in a bioavailability that is improved compared to the bioavailability obtained after administration of the active substance(s) in a plain tablet; or the bioavailability is at least the same or improved compared to the bioavailability obtained after administration of a commercially available product containing the same active substance(s) in the same amounts. In particular, it is desired to obtain quicker and larger and/or more complete uptake of the active compound, and thereby provide for a reduction of the administered dosages or for a reduction in the number of daily administrations.

The terms “compositions,” “pharmaceutical compositions,” “formulations,” and “preparations” are used synonymously and interchangeably herein.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients in pharmaceutical contexts.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that the amount of the compound which, when administered to a subject or patient for treating or preventing a disease, is an amount sufficient to effect such treatment or prevention of the disease. As used herein, the term “IC50” refers to an inhibitory dose which is 50% of the maximum response obtained.

“Prevention” or “preventing” includes: (1) inhibiting or delaying the onset or recurrence of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

The term “eflornithine” when used by itself refers to 2,5-diamino-2-(difluoromethyl) pentanoic acid is any of its forms, including non-salt and salt forms (e.g., eflornithine HCl), anhydrous and hydrate forms of non-salt and salt forms (e.g., eflornithine hydrochloride monohydrate), solvates of non-salt and salts forms, its enantiomers (R and S forms, which may also by identified as d and/forms), and mixtures of these enantiomers (e.g., racemic mixture, or mixtures enriched in one of the enantiomers relative to the other). Specific forms of eflornithine include eflornithine hydrochloride monohydrate (i.e., CAS ID: 96020-91-6; MW: 236.65), eflornithine hydrochloride (i.e., CAS ID: 68278-23-9; MW: 218.63), and free eflornithine (i.e., CAS ID: 70052-12-9; MW: 182.17). Where necessary, the form of eflornithine has been further specified. In some embodiments, the eflornithine of the present disclosure is eflornithine hydrochloride monohydrate (i.e., CAS ID: 96020-91-6). The terms “eflornithine” and “DFMO” are used interchangeably herein. Other synonyms of eflornithine and DFMO include: CPP-1X, α-difluoromethylornithine, 2-(Difluoromethyl)-DL-ornithine, 2-(Difluoromethyl) ornithine, DL-α-difluoromethylornithine, N-Difluoromethylornithine, ornidyl, αδ-Diamino-α-(difluoromethyl) valeric acid, and 2,5-diamino-2(difluro)pentanoic acid.

The term “fixed dose combination” or “FDC” refers to a combination of defined doses of two drugs or active ingredients presented in a single dosage unit (e.g., a tablet or a capsule) and administered as such; further as used herein, “free dose combination” refers to a combination of two drugs or active ingredients administered simultaneously but as two distinct dosage units.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The term “tablet” refers to a pharmacological composition in the form of a small, essentially solid pellet of any shape. Tablet shapes may be cylindrical, spherical, rectangular, capsular or irregular. The term “tablet composition” refers to the substances included in a tablet. A “tablet composition constituent” or “tablet constituent” refers to a compound or substance which is included in a tablet composition. These can include, but are not limited to, the active and any excipients in addition to the low melting compound and the water-soluble excipient.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Treatment of Patients Having Type 1 Diabetes with Eflornithine

The inventors hypothesized that decreasing polyamine synthesis in persons with new onset T1D would decrease polyamine concentrations in urine and serum, and significantly improve markers of β cell health and function, including proinsulin to C-peptide ratios and stimulated C-peptide.

The primary objective of the study was to examine the safety and tolerability of set doses of eflornithine in persons with new onset T1D. The primary efficacy measure was changes in pro-insulin to C-peptide ratios as a marker of β cell ER stress. The secondary objectives of the study were to elucidate the relationship between primary dose and markers of endoplasmic reticulum (ER) stress, polyamine concentrations, glycemic, and immunologic outcomes in persons with new onset T1D, and to characterize the dietary polyamine intake and urinary polyamine excretion of persons with recent-onset T1D. Secondary efficacy measures included other biomarkers of β cell stress, stimulated C-peptide values, and changes in T-cell subsets.

This study was double-blind, placebo-controlled, 2:1 random assigned, phase I/II clinic trial for individuals with type 1 diabetes. The blinded dose-ranging study enrolled persons with new onset T1D with documented continued residual C-peptide production. After a 4 week screening and run-in period during which eligibility was determined and glycemic control optimized, subjects has a 3-month double-masked treatment period with either eflornithine (provided by E. Gerner University of Arizona and Cancer Prevention Pharmaceuticals) or placebo. After a 3 month wash-out period, the durability of effect was assessed.

Patients that were enrolled into the trial had to meet all of the following criteria: 1. Males and females 12-40 years of age with a clinical diagnosis of T1D within 2-8 months of the time of visit 2; 2. Random non-fasting C-peptide level of >0.2 pmol/mL at visit 1; 3. Positive for any one of the following diabetes-related autoantibodies (mIAA, GADA, IA-2A, or ZnT8A); 4. Treatment naïve of any immunomodulatory agent; 5. Normal hearing at screening, defined as acceptable results of pure-tone audiometry (<20 decibel [dB] baseline thresholds for frequencies 250, 500, 1000, and 2000 Hz.

Patients that were enrolled into the trial lacked all of the following criteria: 1. Presence of severe, active disease that interferes with dietary intake or requires the use of chronic medication, with the exception of well-controlled hypothyroidism and mild asthma not requiring oral steroids; 2. Presence of any psychiatric disorder that will affect ability to participate in study, including psychiatric impairment or current use of anti-psychotic medication; 3. Diabetes other than T1D; 4. Chronic illness known to affect glucose metabolism (e.g. Cushing syndrome, polycystic ovarian disorder, cystic fibrosis) or taking medications that affect glucose metabolism (e.g. steroids, metformin); 5. Inability to swallow pills; 6. Hematologic abnormalities at screening (anemia, leukopenia (particularly neutropenia), or thrombocytopenia); 7. Impaired renal function (assessed by history and BUN/Creatinine, eflornithine is renally excreted); 8. Female participants of child-bearing age must not be pregnant and agree to use an effective form of birth control or be abstinent during the study period; 9. BMI >95% for age and sex.

41 subjects were randomly assigned to 1 of 4 sequential dose cohorts. The enrollment cohorts were as follows:

| | Subject | Eflornithine Dose |
|---|---|---|
| Cohort | N (drug/placebo) | mg/m² per day |
| 1 | 9 (6/3) | 125 |
| 2 | 9 (6/3) | 250 |
| 3 | 8 (6/2) | 500 |
| 4 | 9 (7/2) | 750 |
| 5 | 6 (6/0) | 1000 |

The study plan is shown in Table 1.

TABLE 1
Study plan

| | | Treatment | During | Treatment | Follow- | Early |
|---|---|---|---|---|---|---|
| Phase | Screening | Start | Therapy | End | Up | Discontinuation |
| Visit | 1 | 2 | 3 | 4 | 5 | |
| Visit Window | | ~45 days of | +/−3 days | +/−1 week | +/−1 week | |
| | | screen | | | | |
| Informed | X | | | | | |
| consent/Assent | | | | | | |
| Inclusion/Exclusion | X | | | | | |
| criteria | | | | | | |
| Randomization | | X | | | | |
| Eflornithine | | Start | Continue | End | | |
| administration | | | | | | |
| Historical data/ | X | | | | | |
| Demographics* | | | | | | |
| Current medical | X | X | X | X | X | X |
| data** | | | | | | |
| Adverse event | | X | X | X | X | X |
| assessment | | | | | | |
| Dietary | | X | | X | X | X |
| assessment† | | | | | | |
| Audiometric | X | | | X | X | X |
| assessment | | | | | | |
| Detailed physical | X | | | X | | X |
| exam/Height | | | | | | |
| Brief physical | | X | X | | X | |
| exam | | | | | | |
| Weight | X | X | X | X | X | X |
| Vital signs | X | X | X | X | X | X |
| Serum biomarkers | X | X**** | X | X | X | X |
| of ER stress*** | | | | | | |
| Random, non- | X | | | | | |
| fasting C-peptide†† | | | | | | |
| C-peptide by 2-h | | X | | X | X | X |
| MMTT††† | | | | | | |
| Autoantibodies, | X | | | | | |
| Comprehensive | | | | | | |
| metabolic profile | | | | | | |
| HbA1c, CBC, Flow | X | X | X | X | | X |
| cytometry | | | | | | |
| Urine polyamines\hat{ } | | X | | X | | X |
| Eflornithine serum | | | X | X | | X |
| concentrations | | | | | | |
| Pregnancy test | X | X | | X | | X |
| (females only) | | | | | | |

*Historical data: Date of birth, sex, Race/Ethnicity, date of diagnosis of T1D
**Current Medical Data (All visits): Medication use including types of insulin, means of insulin administration, and total daily insulin dose (u/kg body weight, averaged over the 3 days prior to each study visit), use of other medications
****Polymorphisms with DNA were collected and 3 mL of EDTA was also collected at the enrollment visit only
†Dietary Assessments (Visits 2, 4, 5): In order to assess usual diet over the prior 3 months participants provided self-reported dietary information using the Arizona Food Frequency Questionnaire (AFFQ), a validated instrument to estimate polyamine intake. The AFFQ is available for administration both as a paper copy and as a web-based platform. AFFQ measures were collected at three time points across the study. Dietary polyamines were estimated using a food content database. Similarly to adult populations, dietary putrescine was expected to be the major contributor to pediatric polyamine intake. Values for putrescine, spermidine, and spermine were calculated and expressed as nmol/g. Total dietary polyamine was derived by adding these 3 components. The distribution of dietary intakes in the population, as well as the major food contributors, was examined.
***Serum biomarkers of ER Stress: proinsulin, C-peptide, serum for novel proteomics and for biorepository (20 ml)
††Random, non-fasting c-peptide: C-peptide levels were analyzed using a two site immuno-enzymometeric assay using a Tosoh 2000 auto-analyzer (TOSOH, Biosciences, Inc., South San Francisco, CA). The C-peptide assay was calibrated against the WHO IS 84/510 standard and has a sensitivity level of 0.05 ng/mL.
†††C-peptide by 2-h MMTT: 2 hour MMTT was performed using the standardized protocol utilized by the TrialNet Centers. (20 ml blood)
\hat{ }Urine Polyamine Samples: Subjects collected at home first morning urines for measurement of polyamine concentrations. These samples were obtained just prior to the clinic visits (to correspond with the AFFQ measures) and stored in the freezer at home by families until they were transported to the center. This method of urine collection has been used successfully by the PI in other research studies. Once they were received at the center, they were stored at −80° C. until analyzed. High performance liquid chromatography (HPLC) methods were used to detect polyamines as their N-dansylated derivatives. The early discontinuation MMTT was only done if it has been at least 6 weeks since the last MMTT. .

The study participant characteristics at randomization for all groups are shown in Table 2.

TABLE 2
Study participant characteristics at randomization for all groups
(Mean (standard deviation or number (%) as indicated).

| | DFMO dosing group | | | | | | | | | | | |
|---|---|---|---|---|---|---|---|---|---|---|---|---|
| | Placebo | | 125 mg/m² | | 250 mg/m² | | 500 mg/m² | | 750 mg/m² | | 1000 mg/m² | |
| | (n = 10) | | (n = 6) | | (n = 6) | | (n = 6) | | (n = 7) | | (n = 6) | |
| Age, years | 16.5 | (6.5) | 17.0 | (6.3) | 15.0 | (2.7) | 15.5 | (2.6) | 16.2 | (5.3) | 15.7 | (2.3) |

Race

| | | | | | | | | | | | | |
|---|---|---|---|---|---|---|---|---|---|---|---|---|
| Black | 0 | (0.0%) | 0 | (0.0%) | 0 | (0.0%) | 0 | (0.0%) | 1 | (14.3%) | 0 | (0.0%) |
| White | 9 | (90.0%) | 6 | (100%) | 6 | (100%) | 6 | (100%) | 5 | (71.4%) | 6 | (100%) |
| Multiple | 1 | (10.0%) | 0 | (0.0%) | 0 | (0.0%) | 0 | (0.0%) | 1 | (14.3%) | 0 | (0.0%) |
| Female | 5 | (50.0%) | 1 | (16.7%) | 3 | (50.0%) | 1 | (16.7%) | 4 | (57.1%) | 3 | (50.0%) |
| BMI | 22.8 | (2.9) | 21.4 | (2.8) | 22.1 | (4.6) | 27.0 | (7.2) | 23.9 | (4.4) | 21.1 | (3.2) |
| kg/m2 | | | | | | | | | | | | |
| HbA1c % | 7.9 | (1.4) | 7.4 | (1.5) | 6.7 | (1.3) | 6.0 | (0.3) | 6.2 | (0.7) | 7.2 | (1.8) |
| Days since | 156.3 | (63.5) | 166.7 | (71.4) | 125.7 | (58.0) | 148.0 | (68.4) | 128.6 | (50.0) | 86.8 | (29.4) |

T1D
diagnosis

Adverse event (AE) profiles were determined for daily oral doses of 125, 250, 500, 750, and 1000 mg/m² in 41 participants (12-34 YO, 59% male, mean HbAlc 7.3%) with 6-9 participants treated with drug or placebo at each dose. Mild and moderate AEs were noted, including two persons who withdrew, one due to an allergic reaction (diffuse urticaria) and one due to IV access problems. Possible drug-related expected AEs included mild-moderate nausea/vomiting/abdominal pain and diarrhea, moderate headache, upper respiratory infections, a pump site infection, and mild anemia. No unexpected effects judged related to drug occurred in individuals on active drug. Adverse events judges by masked study investigators as possibly or probably related to drug are showing in Table 3.

TABLE 3
Adverse events judged by masked study investigators
as possibly or probably related to drug

| | Dosing Group | | | | | |
|---|---|---|---|---|---|---|
| | | 125 | 250 | 500 | 750 | 1000 |
| | Placebo | mg/m² | mg/m² | mg/m² | mg/m² | mg/m² |
| Adverse Event | (n = 10) | (n = 6) | (n = 6) | (n = 6) | (n = 7) | (n = 6) |
| Gastrointestinal (Total) | 1 | 1 | 1 | 0 | 5 | 2 |
| Nausea | 1 | 0 | 0 | 0 | 3 | 1 |
| Vomiting | 0 | 1 | 0 | 0 | 0 | 0 |
| Diarrhea | 0 | 0 | 1 | 0 | 0 | 0 |
| Abdominal Pain | 1 | 1 | 0 | 0 | 2 | 1 |
| Nausea with IV | 0 | 0 | 0 | 0 | 1 | 0 |
| placement | | | | | | |

Hematologic

| | | | | | | |
|---|---|---|---|---|---|---|
| Grade 1 | 1 | 1 | 0 | 0 | 0 | 0 |
| Neutropenia* | | | | | | |
| Anemia | 2 | 0 | 0 | 0 | 1 | 2 |

Neurologic

| | | | | | | |
|---|---|---|---|---|---|---|
| Headache | 0 | 1 | 0 | 0 | 0 | 2 |
| Dizziness | 0 | 0 | 0 | 0 | 2 | 0 |
| Congestion | 0 | 0 | 0 | 0 | 2 | 1 |
| Cough | 0 | 0 | 0 | 0 | 0 | 1 |
| Hot flashes | 0 | 0 | 0 | 0 | 0 | 1 |
| associated with viral | | | | | | |
| illness | | | | | | |
| URI | 0 | 0 | 0 | 0 | 2 | 2 |
| Other Viral Illness | 0 | 0 | 0 | 0 | 1 | 0 |

Other infectious/immune

| | | | | | | |
|---|---|---|---|---|---|---|
| Pump site infection | 0 | 0 | 0 | 0 | 0 | 1 |

Other

| | | | | | | |
|---|---|---|---|---|---|---|
| Hypoglycemia | 0 | 0 | 0 | 1 | 0 | 0 |
| Urticaria/ | 0 | 0 | 0 | 0 | 1 | 0 |
| anaphylaxis | | | | | | |

*Neutrophil count < Lower limit of normal but above 1500/mm³

Decreases in urinary polyamines were observed with increasing drug dose across groups (Table 4).

TABLE 4
3-month urinary polyamines by drug dose.

| | Drug | Least Square | | |
|---|---|---|---|---|
| | Dose | Means (95% CI) | | P value |
| Putrescine | 0 | 146.5 | μmol/g (95.4, 197.5) | |
| | 125 | 127.9 | μmol/g (63.1, 192.7) | 0.65 |
| | 250 | 78.0 | μmol/g (19.1, 137.0) | 0.08 |
| | 500 | 41.8 | μmol/g (−17.4, 101.0) | 0.01 |
| | 750 | 56.6 | μmol/g (−2.9, 116.1) | 0.03 |
| | 1000 | 35.7 | μmol/g (−33.1, 104.5) | 0.01 |
| Decarboxylated | 0 | 87.5 | μmol/g (54.2, 120.8) | . |
| AdoMet | 125 | 133.0 | μmol/g (90.6, 175.4) | 0.10 |
| | 250 | 87.7 | μmol/g (49.5, 126.0) | 0.99 |
| | 500 | 114.3 | μmol/g (76.0, 152.6) | 0.29 |
| | 750 | 116.0 | μmol/g (77.7, 154.3) | 0.26 |
| | 1000 | 165.7 | μmol/g (123.8, 207.6) | <0.01 |

Compared to the placebo group, individuals receiving the 750 and 1000 mg/m² doses had significantly higher C-peptide area under the curve values at the 6-month post-randomization visit (Table 5).

TABLE 5
3- and 6-month MMTT area under the
curve C-peptide values by drug dose

| | Drug | Least Square | | |
|---|---|---|---|---|
| Timepoint | Dose | Means (95% CI) | | P value |
| 3 months | 0 | 77779.4 | pmol/L (63857.3, 91701.5) | . |
| | 125 | 86367.7 | pmol/L (69073.5, 103662) | 0.43 |
| | 250 | 86172.6 | pmol/L (69109.2, 103236) | 0.44 |
| | 500 | 64785.5 | pmol/L (47546.9, 82024.0) | 0.24 |
| | 750 | 94202.8 | pmol/L (76970.0, 111436) | 0.14 |
| | 1000 | 85819.7 | pmol/L (68802.7, 102837) | 0.46 |
| 6 months | 0 | 68459.5 | pmol/L (53742.1, 83176.9) | . |
| | 125 | 97850.1 | pmol/L (78138.8, 117561) | 0.02 |
| | 250 | 74006.9 | pmol/L (55971.6, 92042.2) | 0.63 |
| | 500 | 66049.5 | pmol/L (47911.3, 84187.6) | 0.84 |
| | 750 | 95403.0 | pmol/L (77270.5, 113535) | 0.03 |
| | 1000 | 95669.7 | pmol/L (77723.8, 113616) | 0.02 |

A 3-month course of oral eflornithine was well tolerated with a favorable AE profile in children and adults with recent-onset T1D and, at higher doses, was associated with greater β cell function compared to placebo.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abeloff M D, Slavik M, Luk G D et al. Phase I trial and pharmacokinetic studies of alpha-difluoromethylornithine—an inhibitor of polyamine biosynthesis. J Clin Oncol 1984; 2(2):124-130.
Bailey H H, Kim K, Verma A K et al. A randomized, double-blind, placebo-controlled phase 3 skin cancer prevention study of {alpha}-difluoromethylornithine in subjects with previous history of skin cancer. Cancer Prev Res (Phila) 2010; 3(1):35-47.
Bardocz S, Duguid T J, Brown D S et al. The importance of dietary polyamines in cell regeneration and growth. Br J Nutr 1995; 73(6):819-828.
Bello-Fernandez C, Packham G, Cleveland J L. The ornithine decarboxylase gene is a transcriptional target of c-Myc. Proc Natl Acad Sci USA. 1993; 90(16):7804-8.
Belting M, Mani K, Jonsson M et al. Glypican-1 is a vehicle for polyamine uptake in mammalian cells: a pivital role for nitrosothiol-derived nitric oxide. J Biol Chem 2003; 278(47):47181-47189.
Belting M, Persson S, Fransson L A. Proteoglycan involvement in polyamine uptake. Biochem J 1999; 338 (Pt 2):317-323.
Bjelakovic G, Beninati S, Bjelakovic B et al. Does polyamine oxidase activity influence the oxidative metabolism of children who suffer of diabetes mellitus? Mol Cell Biochem 2010; 341(1-2):79-85.
Bowlin T L, Mckown B J, Davis G F, Sunkara P S. Effect of polyamine depletion in vivo by DL-alpha-difluoromethylornithine on functionally distinct populations of tumoricidal effector cells in normal and tumor-bearing mice. Cancer Res 1986; 46(11):5494-5498.
Brooks W H. Autoimmune diseases and polyamines. Clin Rev Allergy Immunol 2012; 42(1):58-70.
Celano P, et al. Effect of polyamine depletion on c-myc expression in human colon carcinoma cells. J Biol Chem. 1998; 263(12):5491-4.
Danzin C, Jung M J, Grove J, Bey P. Effect of α-difluoromethylornithine, an enzyme-activated irreversible inhibitor of ornithine decarboxylase, on polyamine levels in rat tissues. Life Sci. 1979; 24:519-524.
DiMeglio L A, Cheng P, Beck R et al. Early Predictors of Stimulated C-peptide in Persons with Type 1 Diabetes (T1D). Abstract. Accepted Poster Presentation at ADA Scientific Sessions 2014.
DiMeglio et al., Changes in beta cell function during the proximate post-diagnosis period in persons with Type 1 Diabetes. Pediatr. Diabetes, 17:237-243, 2016.
Furumitsu Y, Yukioka K, Kojima A et al. Levels of urinary polyamines in patients with rheumatoid arthritis. J Rheumatol 1993; 20(10):1661-1665.
Furumitsu Y, Yukioka K, Yukioka M et al. Interleukin-1beta induces elevation of spermidine/spermine N1-acetyltransferase activity and an increase in the amount of putrescine in synovial adherent cells from patients with rheumatoid arthritis. J Rheumatol 2000; 27(6):1352-1357.
Gerner E W, Meyskens F L, Jr. Combination chemoprevention for colon cancer targeting polyamine synthesis and inflammation. Clin Cancer Res 2009; 15(3):758-761.
Greenbaum C J, Beam C A, Boulware D et al. Fall in C-peptide during first 2 years from diagnosis: evidence of at least two distinct phases from composite Type 1 Diabetes TrialNet data. Diabetes 2012; 61 (8):2066-2073.
Higashi K, Yoshida M, Igarashi A et al. Intense correlation between protein-conjugated acrolein and primary Sjogren's syndrome. Clin Chim Acta 2010; 411(5-6):359-363.
Hong S K, Chaturvedi R, Piazuelo M B et al. Increased expression and cellular localization of spermine oxidase in ulcerative colitis and relationship to disease activity. Inflamm Bowel Dis 2010; 16(9):1557-1566.
Hsu H C, Thomas T, Sigal L H, Thomas T J. Polyamine-fas interactions: inhibition of polyamine biosynthesis in MRL-lpr/lpr mice is associated with the up-regulation of fas mRNA in thymocytes. Autoimmunity 1999; 29(4):299-309.
Jeter J M, Alberts D S. Difluoromethylornithine: the proof is in the polyamines. Cancer Prev Res (Phila) 2012; 5(12):1341-1344.
Maier B, Tersey S A, Mirmira R G. Hypusine: a new target for therapeutic intervention in diabetic inflammation. Discov Med 2010; 10(50):18-23.
Maier B, Ogihara T, Trace A P et al. The unique hypusine modification of eIF5A promotes islet beta cell inflammation and dysfunction in mice. J Clin Invest 2010; 120(6):2156-2170.
McCann P P, Pegg A E (1992) Ornithine decarboxylase as an enzyme target for therapy. Pharmacol Ther 54: 195-215.
Metcalf B W, Bey P, Danzin M J, Casard P & Vevert J P. Catalytic irreversible inhibition of mammalian ornithine decarboxylase (EC 4.1.1.17) by substrate and product analogs. J Amer Chem Soc 1978; 100: 2551-2553.
Meyskens F L, Jr., McLaren C E, Pelot D et al. Difluoromethylornithine plus sulindac for the prevention of sporadic colorectal adenomas: a randomized placebo-controlled, double-blind trial. Cancer Prev Res (Phila) 2008; 1(1):32-38.
Milovic V. Polyamines in the gut lumen: bioavailability and biodistribution. Eur J Gastroenterol Hepatol 2001; 13(9):1021-1025.
Nevins A K, Thurmond D C. Caveolin-1 functions as a novel Cdc42 guanine nucleotide dissociation inhibitor in pancreatic beta-cells. J Biol Chem 2006; 281(28):18961-18972.
Nishiki Y, Adewola A, Hatanaka M, Templin A T, Maier B, Mirmira R G. Translational Control of Inducible Nitric Oxide Synthase by p38 MAPK in Islet beta-Cells. Mol Endocrinol 2013; 27(2):336-349.
O'Shaughnessy J A, Demers L M, Jones S E et al. Alphα-difluoromethylornithine as treatment for metastatic breast cancer patients. Clin Cancer Res 1999; 5(11):3438-3444.
Oredsson S, Anehus S, Heby O: Inhibition of cell proliferation by DL-α-difluoromethylornithine, a catalytic irreversible inhibitor of ornithine decarboxylase. Acta Chem Scan 1980; 34B:457-458.
Packham G, Cleveland J L. The role of ornithine decarboxylase in c-Myc-induced apoptosis. Curr Top Microbiol Immunol 1995; 194:283-290.
Park M H, Nishimura K, Zanelli C F, Valentini S R. Functional significance of eIF5A and its hypusine modification in eukaryotes. Amino Acids 2010; 38(2):491-500.
Pegg, A E, McGovern, K A, & Weist, L. Decarboylation of □-Difluoromethylornithine by ornithine decarboxlyase. Biochem J. 1987; 241, 305-307.
Pena A, et al. Regulation of human ornithine decarboxylase expression by the c-Myc.Max protein complex. J Biol Chem. 1993; 268(36):27277-85.
Pepin J, Milord F, Guern C, Schechter P J. Difluoromethylornithine for arseno-resistant Trypanosoma brucei gambiense sleeping sickness. Lancet 1987; 2(8573):1431-1433.
Poulin, R., Lu, L, Ackermann, B, Bye, P, & Pegg, A. Mechanism of the irreversible inactivation of mouse ornithinedecarboxylase by a-di uoromethylornithine. J. Biol. Chem., 1992; 267, 150-158.
Renaudineau Y, Youinou P. Epigenetics and autoimmunity, with special emphasis on methylation. Keio J Med 2011; 60(1):10-16.
Seiler N. Catabolismof polyamines. Amino Acids 2004; 26(3):217-233.
Selamnia M, Mayeur C, Robert V, Blachier F, alpha]-Difluoromethylornithine (DFMO) as a potent arginase activity inhibitor in human colon carcinoma cells. Biochem Pharmacol. 1998; 55:1241-1245.
Robbins R D, Tersey S A, Ogihara T et al. Inhibition of deoxyhypusine synthase enhances islet {beta} cell function and survival in the setting of endoplasmic reticulum stress and type 2 diabetes. J Biol Chem 2010; 285(51):39943-39952.
Roy U K, Rial N S, Kachel K L, Gerner E W. Activated K-RAS increases polyamine uptake in human colon cancer cells through modulation of caveolar endocytosis. Mol Carcinog 2008; 47(7):538-553.
Sjoholm A, Arkhammar P, Berggren P O, Andersson A. Polyamines in pancreatic islets of obese-hyperglycemic (ob/ob) mice of different ages. Am J Physiol Cell Physiol 2001; 280(2):C317-C323.
Sjoholm A, Arkhammar P, Welsh N et al. Enhanced stimulus-secretion coupling in polyamine-depleted rat insulinoma cells. An effect involving increased cytoplasmic Ca2+, inositol phosphate generation, and phorbol ester sensitivity. J Clin Invest 1993; 92(4):1910-1917.
Soda K, Kano Y, Nakamura T, Kasono K, Kawakami M, Konishi F. Spermine, a natural polyamine, suppresses LFA-1 expression on human lymphocyte. J Immunol 2005; 175(1):237-245.
Soda K, Kano Y, Sakuragi M, Takao K, Lefor A, Konishi F. Long-term oral polyamine intake increases blood polyamine concentrations. J Nutr Sci Vitaminol (Tokyo) 2009; 55(4):361-366.
Tabib A, Bachrach U. Role of polyamines in mediating malignant transformation and oncogene expression. Int J Biochem Cell Biol 1999; 31(11):1289-1295.
Templin A T, Maier B, Nishiki Y, Tersey S A, Mirmira R G. Deoxyhypusine synthase haploinsufficiency attenuates acute cytokine signaling. Cell Cycle 2011; 10(7):1043-1049.
Tersey S A, Nishiki Y, Templin A T et al. Islet beta-cell endoplasmic reticulum stress precedes the onset of type 1 diabetes in the nonobese diabetic mouse model. Diabetes 2012; 61(4):818-827.
Tersey et al., Protective effects of polyamine depletion in mouse models of type 1 diabetes: implications for therapy. Amino Acids 46:633-642, 2014.
Watkins R A, Evans-Molina C, Terrell J et al. Persistence of beta-cell stress in the initial period following diagnosis of T1D in children. Accepted Poster Presentation. ISPAD. 2013.
Welch J E, Bengtson P, Svensson K et al. Single chain fragment anti-heparan sulfate antibody targets the polyamine transport system and attenuates polyamine-dependent cell proliferation. Int J Oncol 2008; 32(4):749-756.
Wolf J E, Jr., Shander D, Huber F et al. Randomized, double-blind clinical evaluation of the efficacy and safety of topical eflornithine HCl 13.9% cream in the treatment of women with facial hair. Int J Dermatol 2007; 46(1):94-98.
Zoumas-Morse C, Rock C L, Quintana E L, Neuhouser M L, Gerner E W, Meyskens F L, Jr. Development of a polyamine database for assessing dietary intake. J Am Diet Assoc 2007; 107(6):1024-1027.

What is claimed is:

1. A method of treating a patient having type 1 diabetes, the method comprising administering to the patient a pharmaceutical therapy comprising an effective amount of eflornithine.

2. The method of claim 1, wherein the method improves β cell health in the patient.

3. The method of claim 1, wherein the method prevents β cell apoptosis in the patient.

4. The method of claim 1, wherein the method preserves residual C-peptide in the patient.

5. The method of claim 1, wherein the method prevents, delays, decreases the likelihood of, or decreases the severity of diabetic ketoacidosis in the patient.

6. The method of claim 1, wherein the method prevents, delays, or slows the progression of severe hypoglycemia in the patient.

7. The method of claim 1, wherein the method prevents, delays, or slows the progression of diabetic nephropathy in the patient.

8. The method of claim 1, wherein the method prevents, delays, or slows the progression of diabetic retinopathy in the patient.

9. The method of any one of claims 1-8, wherein the patient is maintained on a low polyamine diet.

10. The method of any one of claims 1-9, wherein the patient has new onset type 1 diabetes.

11. The method of any one of claims 1-10, wherein the patient was diagnosed with type 1 diabetes no more than eight months before starting treatment with eflornithine.

12. The method of any one of claims 1-11, wherein the patient has not previously received an immunomodulatory agent.

13. The method of any one of claims 1-12, wherein the patient's random C-peptide level is greater than 0.2 pmol/mL.

14. The method of any one of claims 1-13, wherein the patient is an adult.

15. The method of any one of claims 1-13, wherein the patient is a pediatric patient.

16. The method of any one of claims 1-15, wherein the patient's genotype at position +316 (rs2302615) of at least one allele of the ODC1 gene has been determined.

17. The method of any one of claims 1-16, wherein the patient's genotype has been determined to have a G at position +316 (rs2302615) of at least one allele of the ODC1 gene.

18. The method of any one of claims 1-17, wherein the patient's genotype has been determined to have a G at position +316 (rs2302615) of both alleles of the ODC1 gene.

19. The method of any one of claims 1-18, wherein the patient's genotype has been determined to have a G at position +316 (rs2302615) of one allele of the ODC1 gene and an A at position +316 (rs2302615) of one allele of the ODC1 gene.

20. The method of any one of claims 16-19, wherein the method prevents ototoxicity or reduces the risk thereof within the patient.

21. The method of any one of claims 1-20, wherein the eflornithine is eflornithine hydrochloride.

22. The method of claim 21, wherein the eflornithine hydrochloride is eflornithine hydrochloride monohydrate.

23. The method of claim 22, wherein the eflornithine hydrochloride monohydrate is a racemic mixture of its two enantiomers.

24. The method of claim 22, wherein the eflornithine hydrochloride monohydrate is a substantially optically pure preparation.

25. The method of any one of claims 1-24, wherein the eflornithine is administered systemically.

26. The method of claim 25, wherein the eflornithine is administered orally, intraarterially or intravenously.

27. The method of claim 26, wherein the eflornithine is administered orally.

28. The method of claim 27, wherein the effective amount of eflornithine is 125-1500 mg/m²/day.

29. The method of claim 28, wherein the effective amount of eflornithine is 750 mg/m²/day.

30. The method of claim 28, wherein the effective amount of eflornithine is 1000 mg/m²/day.

31. The method of claim 26, wherein the eflornithine is formulated for oral administration.

32. The method of claim 31, wherein the eflornithine is formulated as a hard or soft capsule or a tablet.

33. The method of any one of claims 1-32, wherein the eflornithine is administered every 12 hours.

34. The method of any one of claims 1-32, wherein the eflornithine is administered every 24 hours.

35. The method of any one of claims 1-32, wherein the eflornithine is administered at least a second time.

36. The method of any one of claims 1-35, wherein the patient is human.

37. A composition comprising a pharmaceutically effective amount of eflornithine for use in treating a patient having type 1 diabetes.

38. Use of eflornithine in the manufacture of a medicament for the treatment of type 1 diabetes.
